(19) United States
(12) Patent Application Publication
(10) Pub. No.: US 2022/0375558 A1
(43) Pub. Date: Nov. 24, 2022

(54) SYSTEMS AND METHODS FOR DISPLAYING PATIENT DATA

(71) Applicant: Eli Lilly and Company, Indianapolis, IN (US)

(72) Inventors: Rhett Guy ALDEN, Boston, MA (US); Jennal Lynn JOHNSON, Indianapolis, IN (US); Michelle Lynne KATZ, Newton, MA (US); Howard Allan WOLPERT, Brookline, MA (US)

(21) Appl. No.: 17/772,429

(22) PCT Filed: Nov. 5, 2020
(86) PCT No.: PCT/US2020/059126

§ 371 Date: Apr. 27, 2022

Related U.S. Application Data
(60) Provisional application No. 62/934,080, filed on Nov. 12, 2019.

(51) Int. Cl. G16H 10/60; A61B 5/00; G16H 20/17

(52) U.S. Cl. CPC G16H 10/60; A61B 5/7425; A61B 5/743; G16H 20/17

(57) ABSTRACT

Systems, devices, and methods are provided for displaying selected patient data on a display screen of a computing device. The systems/devices/methods are configured to display on the display screen a plurality of panels, each panel displaying one or more glucose measurements for a patient recorded at different time periods. User input may be received selecting at least one of a plurality of glucose events and at least one of a plurality of contextual factors. In response, the systems/devices/methods may be further configured to display a subset of panels that exhibit each of the selected glucose events and each of the selected contextual factors.

### FIELD OF THE DISCLOSURE

The present disclosure relates to systems, devices and methods for displaying patient data. More particularly, the present disclosure relates to systems, devices, and methods for displaying logged medical and contextual information pertaining to a person with diabetes.

### BACKGROUND OF THE DISCLOSURE

Some persons with diabetes maintain logs and/or records of medical and contextual information pertaining to their condition. Such logs and/or records may include data regarding doses of insulin that they received, such as when such doses were administered and the amount of such doses. Such logs and/or records may also include a historical record of their glucose level measurements. Health care providers may review such records to monitor the person's compliance with their treatment regimen, to detect medical trends or conditions that require treatment, or to identify other issues that require discussion with the person.

### SUMMARY

The present disclosure relates to systems, devices and methods for displaying patient data. Such patient data may include logged medical and contextual information, such as medications taken, blood glucose levels, errors/alerts/information related to insulin delivery devices, information regarding meals (e.g., types of food ingested, amount of food, time of meals), and factors that may affect the patient's health and/or condition (e.g., illnesses, menstruation, stress, exercise/activity, and the like). More particularly, the present disclosure relates to systems, devices and methods for displaying patient data including logged medical and contextual information pertaining to a person with diabetes. Such logged medical and contextual information may include both glucose events and contextual factors such as manual dose overrides, site changes, and/or missed or late boluses.

Various aspects are described in this disclosure, which include, but are not limited to, the following aspects:

1. A method for displaying selected patient data on a display screen of a computing device, the method comprising: displaying, on the display screen of the computing device, a plurality of panels, each panel associated with a unique time window and displaying one or more glucose measurements for a patient recorded during the unique time window; receiving a first user input selecting at least one glucose event type of a plurality of glucose event types; receiving a second user input selecting at least one contextual factor type of a plurality of contextual factor types; and displaying, on the display screen in response to receiving the first and second user inputs, a subset of panels from the plurality of panels that are visually distinguished over other panels from the plurality of panels not included in the subset, wherein each panel in the subset of panels displays at least one glucose measurement that exhibits the selected at least one glucose event type and was recorded during a time period in which the patient experienced the selected at least one contextual factor type.

2. The method of claim 1, further comprising displaying, on the display screen of the computing device, the plurality of glucose event types and the plurality of contextual factor types separate from the plurality of panels.

3. The method of any of claims 1-2, wherein displaying the subset of panels that are visually distinguished over other panels from the plurality of panels not included in the subset comprises visually de-emphasizing panels from the plurality of panels that do not belong to the subset by fading, obscuring, shrinking, or de-saturating the de-emphasized panels on the display screen.

4. The method of any of claims 1-2, wherein displaying the subset of panels that are visually distinguished over other panels from the plurality of panels not included in the subset comprises removing, from the display screen in response to receiving the first and second user inputs, panels from the plurality of panels that do not belong to the subset of panels.

5. The method of any of claims 1-4, wherein displaying the subset of panels that are visually distinguished over other panels from the plurality of panels not included in the subset comprises visually emphasizing the subset of panels on the display screen by changing a color of said subset of panels, adding a border around said subset of panels, adding a symbol to each panel of said subset of panels, or increasing a size of each panel of said subset of panels.

6. The method of any of claims 1-5, wherein the plurality of glucose events comprise at least one of a hypoglygemic event, a nocturnal hypoglycemic event, a hyperglycemic event, and a prolonged hyperglycemic event.

7. The method of any of claims 1-6, wherein the plurality of contextual factors comprise at least one of a user override of an automatic dose increase, a user override of an automatic dose decrease, a late bolus, a manual bolus, a missed bolus, a critical pump alarm, a change of an infusion site, and a suspension of an automatic infusion dosing algorithm.

8. The method of any of claims 1-7, wherein each panel of the plurality of panels displays one or more glucose measurements for a patient recorded during a different day.

9. The method of any of claims 1-8, wherein each panel displays one or more glucose measurements recorded by at least one of a blood glucose monitor (BGM), a continuous glucose monitor (CGM), and a flash glucose monitor (FGM).

10. A computing device comprising: a display screen; at least one processor; and non-transitory computer-readable media storing computer-executable instructions that, when executed by the at least one processor, are operable to cause the at least one processor to: display on the display screen a plurality of panels, each panel associated with a unique time window and displaying one or more glucose measurements for a patient recorded during the unique time window; receive a first user input selecting at least one glucose event type of a plurality of glucose event types; receive a second user input selecting at least one contextual factor type of a plurality of contextual factor types; and display, on the display screen in response to receiving the first and second user inputs, a subset of panels from the plurality of panels that are visually distinguished over other panels from the plurality of panels not included in the subset, wherein each panel in the subset of panels displays at least one glucose measurement that exhibits the selected at least one glucose event type and was recorded during a time period in which the patient experienced the selected at least one contextual factor type.

11. The computing device of claim 10, wherein the at least one processor is further configured to display, on the display screen of the computing device, the plurality of glucose event types and the plurality of contextual factor types separate from the plurality of panels.

12. The computing device of any of claims 10-11, wherein displaying the subset of panels that are visually distinguished over other panels from the plurality of panels not included in the subset comprises visually de-emphasizing panels from the plurality of panels that do not belong to the subset by fading, obscuring, shrinking, or de-saturating the de-emphasized panels on the display screen.

13. The computing device of any of claims 10-11, wherein displaying the subset of panels that are visually distinguished over other panels from the plurality of panels not included in the subset comprises removing, from the display screen in response to receiving the first and second user inputs, panels from the plurality of panels that do not belong to the subset of panels.

14. The computing device of any of claims 10-13, wherein displaying the subset of panels that are visually distinguished over other panels from the plurality of panels not included in the subset comprises visually emphasizing the subset of panels on the display screen by changing a color of said subset of panels, adding a border around said subset of panels, adding a symbol to each panel of said subset of panels, or increasing a size of each panel of said subset of panels.

15. The computing device of any of claims 10-14, wherein the plurality of glucose events comprise at least one of a hypoglygemic event, a nocturnal hypoglycemic event, a hyperglycemic event, and a prolonged hyperglycemic event.

16. The computing device of any of claims 10-15, wherein the plurality of contextual factors comprise at least one of a user override of an automatic dose increase, a user override of an automatic dose decrease, a late bolus, a manual bolus, a missed bolus, a critical pump alarm, a change of an infusion site, and a suspension of an automatic infusion dosing algorithm.

17. The computing device of any of claims 10-16, wherein each panel of the plurality of panels displays one or more glucose measurements for a patient recorded during a different day.

18. The computing device of any of claims 10-17, wherein each panel displays one or more glucose measurements recorded by at least one of a blood glucose monitor (BGM), a continuous glucose monitor (CGM), and a flash glucose monitor (FGM).

19. Non-transitory computer-readable media storing computer-executable instructions that, when executed by one or more processors, are operable to cause the one or more processors to: display, on a display screen of a computing device, a plurality of panels, each panel associated with a unique time window and displaying one or more glucose measurements for a patient recorded during the unique time window; receive a first user input selecting at least one glucose event type of a plurality of glucose event types; receive a second user input selecting at least one contextual factor type of a plurality of contextual factor types; and display, on the display screen in response to receiving the first and second user inputs, a subset of panels from the plurality of panels that are visually distinguished over other panels from the plurality of panels not included in the subset, wherein each panel in the subset of panels displays at least one glucose measurement that exhibits the selected at least one glucose event type and was recorded during a time period in which the patient experienced the selected at least one contextual factor type.

20. The non-transitory computer-readable media of claim 19, wherein the computer-executable instructions, when executed by the one or more processors, are further operable to cause the one or more processors to display, on the display screen of the computing device, the plurality of glucose event types and the plurality of contextual factor types separate from the plurality of panels.

21. The non-transitory computer-readable media of any of claims 19-20, wherein displaying the subset of panels that are visually distinguished over other panels from the plurality of panels not included in the subset comprises visually de-emphasizing panels from the plurality of panels that do not belong to the subset by fading, obscuring, shrinking, or de-saturating the de-emphasized panels on the display screen.

22. The non-transitory computer-readable media of any of claims 19-20, wherein displaying the subset of panels that are visually distinguished over other panels from the plurality of panels not included in the subset comprises removing, from the display screen in response to receiving the first and second user inputs, panels from the plurality of panels that do not belong to the subset of panels.

23. The non-transitory computer-readable media of any of claims 19-22, wherein displaying the subset of panels that are visually distinguished over other panels from the plurality of panels not included in the subset comprises visually emphasizing the subset of panels on the display screen by changing a color of said subset of panels, adding a border around said subset of panels, adding a symbol to each panel of said subset of panels, or increasing a size of each panel of said subset of panels.

24. The non-transitory computer-readable media of any of claims 19-23, wherein the plurality of glucose events comprise at least one of a hypoglygemic event, a nocturnal hypoglycemic event, a hyperglycemic event, and a prolonged hyperglycemic event.

25. The non-transitory computer-readable media of any of claims 19-24, wherein the plurality of contextual factors comprise at least one of a user override of an automatic dose increase, a user override of an automatic dose decrease, a late bolus, a manual bolus, a missed bolus, a critical pump alarm, a change of an infusion site, and a suspension of an automatic infusion dosing algorithm.

26. The non-transitory computer-readable media of any of claims 19-25, wherein each panel of the plurality of panels displays one or more glucose measurements for a patient recorded during a different day.

27. The non-transitory computer-readable media of any of claims 19-26, wherein each panel displays one or more glucose measurements recorded by at least one of a blood glucose monitor (BGM), a continuous glucose monitor (CGM), and a flash glucose monitor (FGM).

### BRIEF DESCRIPTION OF THE DRAWINGS

The above-mentioned and other features and advantages of this disclosure, and the manner of attaining them, will become more apparent and will be better understood by reference to the following description of embodiments of the invention taken in conjunction with the accompanying drawings, wherein:

FIG. 1 depicts an exemplary communication system 100, according to some embodiments.

FIG. 2 depicts an illustrative implementation of a computer system 200 that may be used to perform any of the methods and embodiments disclosed herein, according to some embodiments.

FIG. 3 is a flowchart depicting an exemplary process 300 for displaying patient data pertaining to a person with diabetes, according to some embodiments.

FIG. 4 illustrates one example of a display screen having a plurality of panels including glucose measurements for the person with diabetes, according to some embodiments.

FIG. 5 illustrates a close-up view of one of the panels displayed in FIG. 4, according to some embodiments.

FIG. 6 illustrates an alternative version of the panel displayed in FIG. 5 including detailed patient data, according to some embodiments.

FIG. 7 shows an example display screen in which a user has selected the glucose event “hypo” (e.g., hypoglycemic event), according to some embodiments.

FIG. 8 shows an example display screen in which the user has selected both the glucose event “hypo” (e.g., hypoglycemic event) and the contextual factor “upward dose override”, according to some embodiments.

FIG. 9 shows an example panel for comparing logged medical and/or contextual data from multiple time periods, according to some embodiments.

Corresponding reference characters indicate corresponding parts throughout the several views. The exemplifications set out herein illustrate exemplary embodiments of the invention and such exemplifications are not to be construed as limiting the scope of the invention in any manner.

### DETAILED DESCRIPTION

Persons with diabetes may maintain logs and/or records of the doses of insulin that they received, as well as of their glucose level measurements. Such logs and/or records may also sometimes be supplemented with contextual factors. Such contextual factors can include whether the dose they received was calculated by a bolus calculator, and if so, whether the dose they received was higher or lower than the dose recommended by the calculator. Such contextual factors may also include data regarding whether and/or when the person missed a bolus or took a bolus late, whether and/or when an automatic insulin delivery device of the person experienced a critical failure (e.g., an occlusion), or whether and/or when the person changed an infusion site of the automatic insulin delivery device.

Health care providers may review such logs and/or records with the person to monitor the person's compliance with their treatment regimen, to detect medical trends or conditions that require treatment, or to identify other issues that require discussion with the person. However, as the amount of information contained in the person's log and/or records increases, health care providers (HCPs) may find it increasingly difficult to quickly accomplish these tasks. Accordingly, there is a need to provide a user interface that quickly allows HCPs to filter and sort through the person's records to identify medical issues that require discussion or treatment.

Furthermore, correlations between logged events may sometimes indicate deeper issues that require discussion or treatment, or may reveal insights into the root causes of persistent medical issues. For example, a consistent trend of undesirably high glucose levels (e.g., a relatively high number of hyperglycemic events) in a person's glucose record may be due to a large number of factors. Without pinpointing the root cause of such high glucose levels, a HCP may not be able to counsel the person effectively on how to avoid such hyperglycemic events in the future.

However, if the HCP observes that the person's hyperglycemic events tend to be correlated with days where the person manually overrides a calculated bolus dose downward (i.e., a downward dose override), the HCP may reasonably infer that the person suffers from a fear of hypoglycemia. In this scenario, the HCP may refer the person to education and/or resources that help the person overcome his/her fear of hypoglycemia and gain better judgment on when to override a bolus dose suggested by his/her bolus calculator.

On the other hand, if the HCP observes that the person's hyperglycemic events tend to be correlated with days where the person's automated insulin delivery device has a critical low insulin reservoir alarm (e.g., the device is running out of insulin), the HCP may reasonably infer that the person tends not to refill his/her insulin delivery device often enough. In this scenario, the proper course of action may be to better educate the person on how to maintain and/or refill his/her insulin delivery device, or potentially to inquire into whether the person requires financial assistance in obtaining needed insulin.

As yet another example, if the HCP observes that the person's hyperglycemic events tend to be correlated with days where the person has a missed bolus or a late bolus, the HCP may reasonably infer that the person has trouble remembering to take a bolus of insulin with every meal. Indeed, if the person's records indicate the person has no trouble taking a bolus at dinner time but tends to have trouble taking a bolus at lunch time, the HCP may infer that the person has difficulty taking a bolus at his/her work place in particular. In this scenario, the proper course of action may be to work with the person to devise a system or to form habits that help the person take boluses with every meal.

As this simple example illustrates, a single glucose event (e.g., a hyperglycemic event) may be caused by different root causes, each of which may be best addressed using different strategies. By correlating observed glucose events with contextual factors, HCPs may derive valuable insights into the root causes behind such glucose events and thereby devise effective strategies in preventing such glucose events in the future. Accordingly, there is a need to provide a user interface that quickly allows HCPs to correlate observed glucose events (e.g., hypoglycemic or hyperglycemic events) with contextual factors (e.g., manual dose overrides, or critical alarms related to the person's insulin delivery device). The systems, methods, and apparatus disclosed herein are configured to help address at least some of the above-described needs.

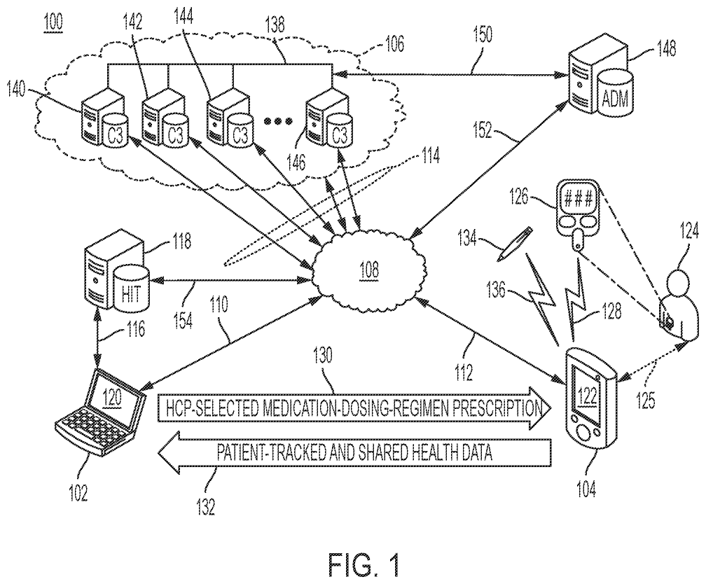

FIG. 1 depicts a communication system 100 that includes an example HCP system 102, an example health IT system 118, an example mobile device 104 associated with an example patient 124 (e.g., a person with diabetes) and in communication with multiple example personal medical devices (in this depiction, an example glucose meter 126 and an example connected injection device 134) that are also associated with the example patient 124, an example server system 106 that includes one or more example servers 140-146 that are communicatively interconnected with one another via an example private network 138, and an example administrator-portal system 148, in accordance with at least one embodiment. Also depicted in FIG. 1 are a data network 108, communication links 110, 112, 114, 116, 128, 136, 150, 152, and 154; a display 120 of the HCP system 102; and a display 122 of the mobile device 104. Further displayed are an association arrow 125, a conceptual-information-flow arrow 130, and a conceptual-information-flow arrow 132, both of which are described below. It should be understood that the entities and the arrangement and interconnection thereof that are depicted in FIG. 1 are provided for illustration and by way of example and not limitation, and that other entities, arrangements, and interconnections could be implemented as well as deemed suitable by those of skill in the art for a given context.

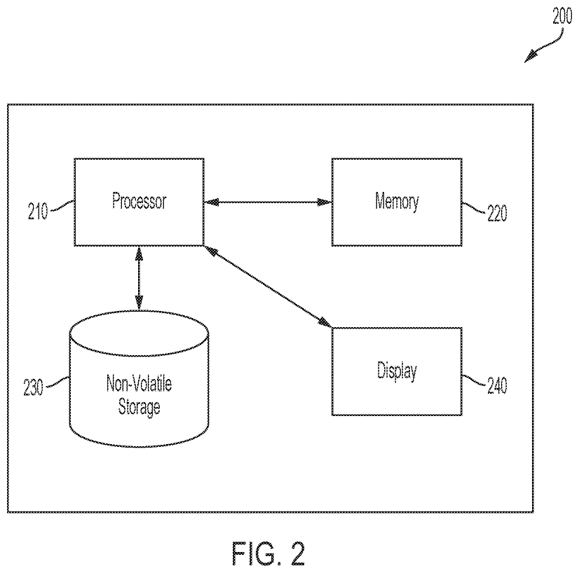

The HCP system 102 (including the display 120) is described from a physical-architecture standpoint more fully below in connection with FIG. 2, but in general may take the form of any computing device that is equipped, configured, and programmed to carry out the functions described herein. Some options for the HCP system 102 include a desktop computer, a laptop computer, a tablet computer, a mobile device such as a smartphone, or a device that interacts with the HCP using voice, augmented reality (AR) or virtual reality (VR). In some embodiments, HCP system 102 is any electronic device capable of supporting and running an Internet browser (i.e., a web browser—perhaps as a standalone application, perhaps as a capability of another application, to name but a few examples). As depicted in FIG. 1, the HCP system 102 is communicatively connected to the health IT system 118 via the communication link 116 and to the data network 108 via the communication link 110. Although communication link 116 is depicted as being separate from data network 108, it should be understood that in some instances, all or part of link 116 may traverse at least a portion of data network 108. In some instances, the HCP system 102 may communicate with HIT system 118 indirectly via the data network 108, instead of through a direct link 116 as depicted in FIG. 1. Among the typical users of the HCP system 102 are HCPs with prescribing roles (e.g., doctors, physician assistants (PAs), nurse practitioners, and the like), as well as non-prescribing HCPs who are part of a care team, and users associated with integrated delivery networks (IDNs), among other examples.

In various different embodiments, the HCP system 102 may implement a HCP portal application or interface that provides and supports functions such as prescribing medication-dosing regimens (e.g., insulin-dosing regimens such as basal-insulin-dosing regimens, bolus-insulin-dosing regimens, and/or the like), modifying existing dosing-regimen prescriptions, setting and/or modifying dosing-regimen parameters, and viewing individual patient data, among other examples that could be listed here. The prescription and parameter-setting with respect to dosing regimens by an HCP via the HCP system 102 assists patients in determining medication doses. Among other functions, the HCP system 102 provides the HCP with the capability to download patient EHR data from the health IT system 118 via the communication link 116 and view the EHR data in the HCP portal, view a selection of available medication-dosing regimens, select a dosing regimen to prescribe to the patient, and assign values to required parameters for the selected dosing regimen (such as starting dose, insulin-to-carb ratio, and the like, in the example case of an insulin-dosing regimen). The HCP portal on the HCP system 102 further provides data-visualization features that enable the HCP users to review patient historical data, as described in further detail below. In various embodiments, this patient historical data is viewable in chart form, graphical form, tabular form, and/or one or more other forms deemed suitable by those of skill in the art for a given implementation.

The mobile device 104 (including the display 122) is described from a physical-architecture standpoint more fully below in connection with FIG. 2, but in general could take the form of any computing device that is equipped, configured, and programmed to carry out the mobile-device functions described herein. Some options for the mobile device 104 include a cell phone, a smart phone, a personal digital assistant (PDA), a tablet computer, a laptop computer, a wearable device, a mobile device that interacts with its user via voice or virtual reality, and the like. As depicted in FIG. 1, the mobile device 104 is communicatively connected to the glucose meter 126 via the communication link 128, to the connected injection device 134 via the communication link 136, and to the data network 108 via the communication link 112. As is depicted by the double-ended-and-dashed association arrow 125, the mobile device 104 is associated—e.g., by a subscription account, ownership, possession, and/or in one or more other ways—with the patient 124.

With respect to the C3-server system 106, an example one of the C3 servers 140-146 is described from a physical-architecture standpoint more fully below in connection with FIG. 2. In general, however, the C3-server system 106 could take the form of any set of one or more servers that are collectively equipped, configured, and programmed to collectively carry out the C3-server-system functions described herein. As depicted in FIG. 1, the C3-server system 106 is communicatively connected to the data network 108 via the communication link 114.

It is noted that, as depicted in FIG. 1, each respective C3 server 140-146 in the C3-server system 106 could have its own respective communication link 114 with the data network 108; also or instead, a single communication link 114 could communicatively connect the C3-server system 106 with the data network 108, perhaps via a firewall, a network access server (NAS), and/or the like. For clarity of presentation, the one or more communication links 114 are referred to as “the communication link 114” in the balance of this disclosure. Moreover, as is also depicted in FIG. 1, the respective C3 servers 140-146 are communicatively interconnected with one another via a private network 138, which could take the form of or include a local area network (LAN), a virtual private network (VPN), and/or one or more other options deemed suitable by those of skill in the art for a given implementation. In some embodiments, the depicted network 138 does not exist as a separate network as currently depicted—in such embodiments, the respective C3 servers 140-146 communicate with one another via their links to data network 108. The communicative connectivity between the C3-server system 106 and the administrator-portal system 148 is discussed below.

As a general matter, the C3-server system 106 functions as “the cloud” as that term is used in the art with respect to entities such as the HCP system 102, the health IT system 118, and the mobile device 104 (and in particular to one or more applications executing on the mobile device 104). In some embodiments, a subset of C3 servers 140-146 may be dedicated to serving at least one of the HCP system 102, the health IT system 118, and the mobile device 104. In some embodiments, however, each of the C3 servers 140-146 may be capable of serving any of these three entities. Regardless of the specific architectural implementation, the C3-server system 106 functions as at least one cloud with particular purposes for those entities. As such, in at least one embodiment, all communication to and from the C3-server system 106 with any one or more other entities is secure communication—as examples, such communication could be encrypted and/or signed as is known in the art. Such communication could be inside a tunnel such as a VPN, among other communication-security and data-security options that could be implemented as deemed suitable by those of skill in the art in various contexts.

In various different embodiments, and as is further described below, the C3-server system 106 provides and supports functions—for the mentioned entities and perhaps others-such as secure and reliable transfer of information and data (related to, e.g., prescriptions, patient-tracked and shared health data) between the HCP system 102 and applications running on mobile devices associated with patients, data storage, management of relationships between patients and HCPs, Integrated Delivery Networks (IDNs, e.g., a network of health care organizations), and the like, and in some embodiments, instead or in addition, provides and supports one or more other functions deemed suitable by those of skill in the art for a given implementation. Moreover, in some embodiments, the C3-server system 106 facilitates data sharing that involves payers (e.g., insurance companies); in some such embodiments, such data sharing with payer entities is conditional upon the associated patients opting in to allow such communication. And other examples of provided and supported functions could be listed here as well.

The administrator-portal system 148 is described from a physical-architecture standpoint more fully below in connection with FIG. 2 but in general could take the form of any computing device that is equipped, configured, and programmed to carry out the administrator-portal-system functions described herein. Some options for the administrator-portal system 148 include a desktop computer, a laptop computer, a tablet computer, a workstation, and the like. As depicted in FIG. 1, the administrator-portal system 148 in at least one embodiment is operable to communicate with the C3-server system 106 (and in particular in the depicted example via the private network 138) via the communication link 150. In other embodiments, as is also depicted in FIG. 1, the administrator-portal system 148 is operable to communicate with the HCP system 102, the mobile device 104, and/or the C3-server system 106 via the data network 108. And in some embodiments, the administrator-portal system 148 is operable to do both.

As a general matter, in various different embodiments, the administrator-portal system 148 provides various services with respect to the HCP-portal 102, an application (e.g., a Mobile Medical Application, or MMA) executing on the mobile device 104, and/or the C3-server system 106. One example category of such services are those that pertain to user management, login, access level, and the like. In at least one embodiment, a user with sufficient permissions can use the administrator-portal system 148 to change and/or manage various settings of the HCP-portal 102, an MMA executing on the mobile device 104, and/or the C3-server system 106. In some cases, changes made via the administrator-portal system 148 affect only a single MMA, a single user, a single HCP, and/or the like; in other cases, such changes affect multiple MMAs, multiple user accounts, multiple HCPs, and/or the like. For example, an IDN may be provided with an administrator-portal system 148 that governs accounts of patients enrolled in the IDN. In some embodiments, the administrator-portal system 148 is operable to roll out updates, upgrades, and the like. In some embodiments, the administrator-portal system 148 is operable to manage individual HCP accounts, individual patient accounts, and/or the like. And certainly numerous other example administrative functions for which the administrator-portal system 148 could be used could be listed here.

In the example environment that is depicted in FIG. 1, the data network 108 is communicatively connected with the HCP system 102 via the communication link 110, with the mobile device 104 via the communication link 112, with the C3-server system 106 via the communication link(s) 114, and with the administrator-portal system 148 via the communication link 152. In at least one example scenario, the data network 108 includes one or more Internet Protocol (IP) networks such as the worldwide network of networks typically referred to broadly as the Internet, one or more public IP networks, one or more private (e.g., corporate) IP networks, and/or one or more IP networks of any other type deemed suitable by those of skill in the art for a given implementation. Entities that communicate via the data network 108 may be identified by an address such as an IP address (e.g., an IPv4 address or an IPv6 address). It is not required that the data network 108 be or include an IP network, however, as this is merely an example.

As used herein, a “communication link” includes one or more wired-communication (e.g., Ethernet, Universal Serial Bus (USB), and/or the like) links and/or one or more wireless-communication (e.g., cellular, Wi-Fi, Bluetooth, and/or the like), and may also include any suitable number of relay devices such as routers, switches, bridges, and/or the like. The communication link 112 in particular may include at least one wireless-communication link to facilitate two-way data communication with the mobile device 104. Moreover, either or both of the communication links 128 and 136 may take the form of or at least include at least one of a near-field communication (NFC) link, a Bluetooth link, a radio-frequency identification (RFID) link, a direct radio frequency (RF) link, and/or one or more other types of wireless-communication links. Moreover, the communication links 128 and 136 could but need not be point-to-point links between (i) the mobile device 104 and (ii) the glucose meter 126 and connected injection device 134, respectively: in some embodiments, one or both of the communication links 128 and 136 are an indirect link via, e.g., a Wi-Fi or ZigBee router, or a cellular network or tower (not depicted). And other implementation examples could certainly be listed here as well.

In the example scenario that is depicted in FIG. 1, the health IT system 118 is communicatively connected to the HCP system 102 via the communication link 116, and in general could take the form of one or more servers. The health IT system 118 may or may not have its own local user interface, such as its own monitor display, keyboard, mouse, touchscreen, or other user interface. In various different embodiments, the health IT system 118 optionally provides and supports secure maintenance of, secure storage of, and secure access to patient Electronic Health Records (EHRs—also referred to as Electronic Medical Records or EMRs in the industry), perhaps among other functions deemed suitable for a health IT system by those of skill in the art. Moreover, health IT system 118 is communicatively coupled to data network 108 via communication link 154. Health IT system 118 may also communicate with C3-server system 106 via data network 108. In some embodiments, the health IT system 118 interfaces with the C3-server system 106 and/or HCP system 102 via a standardized protocol, such as the Fast Healthcare Interoperability Resources (FHIR) protocol, or the Substitutable Medical Applications and Reusable Technologies (SMART) protocol, as examples.

In the example scenarios described herein, the patient 124 has been diagnosed with diabetes and is being treated by an HCP that is associated with the HCP system 102, though this is purely by way of example and not limitation. In the described embodiments, both the mobile device 104 (and at least one MMA running thereon), the glucose meter 126, and the connected injection device 134 are each associated with the patient 124, and in at least that way with one another. The above-mentioned association arrow 125 is intended to represent a general association and a user-interface-level interaction of the patient 124 with the mobile device 104.

It is further noted that while FIG. 1 shows mobile device 104 being communicatively connected to both glucose meter 126 and connected injection device 134, in some cases, only one of the glucose meter 126 and the connected injection device 134 may be present in various different scenarios, and that both are not required. Indeed, some scenarios involve no additional medical devices connected to mobile device 104 at all, such as is the case with systems that require patients to separately and manually measure glucose levels and/or log dosage information. In some cases, a patient may connect more than one glucose meter, and/or more than one connected injection device, to the MMA of the mobile device 104—for example, a patient may have one glucose meter and/or injection device at home, and another glucose meter and/or injection device at work. The MMA may be configured to support multiple connections of each. Moreover, in some cases, one or more connected medical devices other than a glucose meter and/or a connected injection device are present. Some examples include a blood-pressure monitor, a pulse/oxygen monitor, and/or other suitable medical devices.

The glucose meter 126 is associated with the patient 124 for medical, diabetes-treatment purposes, and is communicatively connected with the mobile device 104 via the above-described communication link 128. The glucose meter 126 could include a blood-glucose meter (BGM), a continuous glucose meter (CGM), a flash glucose monitor (FGM), or other devices that measure the patient 124's blood glucose or other sources of glucose levels (e.g., contact lens devices, or devices that use near IR radiation to measure glucose levels). A BGM takes discrete spot measurements of the patient's blood-glucose level (e.g., by pricking the patient's finger to conduct spot measurements of the patient's capillary whole blood glucose level). Both CGM and FGM use sensors to measure interstitial glucose. A CGM system may include a sensor, transmitter and receiver/app. A CGM may take more frequent (i.e., more continuous) measurements of the patient's interstitial glucose levels and may optionally be continuously worn by the patient for relatively extended periods of time (e.g., several hours or days at a time). One example of such a CGM is the G6 sensor manufactured by Dexcom, Inc. A FGM system may comprise a sensor worn on or inserted into a portion of the patient's body, and a reader (e.g., a handheld reader) that, when activated or when placed in close proximity to the sensor, receives a glucose level reading from the sensor. One example of such a FGM is the FreeStyle Libre device, manufactured by Abbott Diabetes Care Inc. In some embodiments, a FGM does not require finger-stick calibration. Other types of glucose meters may be provided as well.

CGM and FGM systems may measure interstitial glucose levels, while BGM systems may measure blood glucose levels. For simplicity and readability, this disclosure refers simply to a “glucose” level, or “GL.” It is understood that such references may refer to either blood glucose or interstitial glucose, as appropriate.

The connected injection device 134 is also associated with the patient 124 for medical, diabetes-treatment purposes, and is communicatively connected with the mobile device 104 via the above-described communication link 136. Device 134 may further comprise a drug or medication. In some embodiments, a system may comprise one or more devices including device 134 and a drug or medication. The term “drug” or “medication” refers to one or more therapeutic agents including but not limited to insulins, insulin analogs such as insulin lispro or insulin glargine, insulin derivatives, glucagon, glucagon analogs, glucagon derivatives, and any therapeutic agent that is capable of delivery by the above device. The drug or medication as used in the device 134 may be formulated with one or more excipients. The device is operated in a manner generally as described herein by a patient, caregiver or healthcare professional to deliver the drug to a person.

In at least one embodiment, the connected injection device 134 is or at least includes what is referred to at times in the art as a connected insulin-delivery device (e.g., a connected insulin pen, such as a pen having integrated and/or attachable electronics to auto-detect and report to another electronic device an amount of injected insulin). In various different embodiments, the connected injection device 134 takes the form of or includes one or more of the insulin-delivery devices described in any of the following patent applications, each of which is hereby incorporated herein by reference in its respective entirety:
PCT Application No. PCT/US17/65251 filed Dec. 8, 2017 and entitled Medication Delivery Device with Sensing System (Attorney Docket No. X21353);
PCT Application No. PCT/US18/19156 filed Feb. 22, 2018 and entitled Dose Detection and Drug Identification for a Medication Delivery Device (Attorney Docket No. X21457); and
PCT Application No. PCT/US18/47442 filed Aug. 22, 2018 and entitled Dose Detection with Piezoelectric Sensing for a Medication Delivery Device (Attorney Docket No. X21462).

In some embodiments, the connected injection device 134 takes the form of an automated insulin delivery device, such as an insulin pump. Such automated insulin delivery devices may include a reservoir sized to carry sufficient basal and/or bolus insulin for multiple doses and may be configured to be worn on or attached to the patient 124's body. The device may automatically infuse such basal and/or bolus insulin into the patient's body via an infusion set attached to the patient's body, e.g., the patient's abdomen, back, or arm. One example of an automated insulin delivery device is the MiniMed™ 670G Insulin Pump System, manufactured and sold by Medtronic PLC. In yet other embodiments, the mobile device 104 may be communicably coupled to two or more injection devices 134, such as both a connected insulin pen as well as an automated insulin delivery device.

Moreover, in some cases, one or more of the capabilities of one of those two devices in this disclosure are also or instead covered by the other of those two devices and/or by one or more additional devices. In one example, a single device can both monitor glucose (and report back results) and inject insulin (and report back injected amounts). And certainly other combinations of capabilities could be listed here.

Furthermore, and as also described below, in some embodiments, an MMA executing on the mobile device 104 communicates for various reasons (e.g., sending dosing commands, receiving dosed-amount confirmation reports, requesting (e.g., glucose) readings, receiving one or more measured values, and/or the like) with one or more connected medical devices such as the example glucose meter 126 and connected injection device 134. Such communication may be in one-way or two-way manner with a given device. Additional examples of information that could be conveyed from a connected medical device to an MMA include error codes, device metrics, dosing records, and/or dosing confirmations. And certainly other examples could be listed here. Moreover, in some cases, direct communication links exist between various connected medical devices, such as between the glucose meter 126 and the connected injection device 134.

The conceptual-information-flow arrow 130 is meant to graphically and conceptually depict that, among other information and as is described more fully below, the HCP system 102 may transmit, either directly, via data network 108, via the C3-server system 106, or via some other intermediate component or network, HCP-selected medication-dosing-regimen (e.g., bolus-dosing-regimen) prescriptions to the MMA executing on the mobile device 104. Similarly, the conceptual-information-flow arrow 132 is meant to graphically and conceptually depict that, among other information and as is described more fully below, the MMA executing on the mobile device 104 may transmit, either directly or via the C3-server system 106, patient-tracked and patient-shared health data regarding the patient's diabetes and/or one or more other health-related conditions, topics, and the like.

As a general matter, it should be understood that any of the entities described herein, including but not limited to the HCP system 102, the health IT system 118, the mobile device 104 (including an MMA executing thereon), the C3-server system 106, and the administrator-portal system 148—may communicate in at least one embodiment with any other of those entities without being required to route that communication through one or more other entities. For example, in at least one embodiment, the HCP system 102 and the mobile device 104 can exchange information without that information having to pass through the C3-server system 106. In some embodiments, however, one or more entities communicate with one another via at least one additional entity; as an example, in at least one embodiment, data (e.g., HCP-selected-regimen data) is communicated from the HCP system 102 to the MMA executing on the mobile device 104 via the C3-server system 106.

Further details regarding and exemplary embodiments of communication context 100 are described in International App. No. PCT/US19/42507, filed Jul. 19, 2019 and entitled SYSTEMS AND METHODS FOR REMOTE PRESCRIPTION OF MEDICATION-DOSING REGIMENS, the entire contents of which are incorporated herein by reference.

An illustrative implementation of a computer system 200 that may be used to perform any of the aspects of the methods/processes and embodiments disclosed herein is shown in FIG. 2. The computer system 200 may be the general computer architecture for any or all of the previously-described systems and devices, such as HCP system 102, health IT system 118, mobile device 104, C3 servers 140-146, and/or administrator-portal system 148. The computer system 200 may include one or more processors 210 and one or more non-transitory computer-readable storage media (e.g., memory 220 and one or more non-volatile storage media 230) and an optional display 240. The processor 210 may control writing data to and reading data from the memory 220 and the non-volatile storage device 230 in any suitable manner, as the aspects of the invention described herein are not limited in this respect. To perform functionality and/or techniques described herein, the processor 210 may execute one or more instructions stored in one or more computer-readable storage media (e.g., the memory 220, storage media, etc.), which may serve as non-transitory computer-readable storage media storing instructions for execution by the processor 210. The optional display 240 may in some embodiments comprise a graphical user interface comprising a touchscreen display operative to receive user inputs. The computer system 200 may also include one or more other data input devices, such as a computer keyboard or mouse, stylus, voice input device (e.g., microphone), camera, and the like.

In connection with techniques described herein, software or firmware code used to, for example, display logged data pertaining to a person with diabetes may be stored on one or more computer-readable storage media of computer system 200. Processor 210 may execute any such code to provide any techniques for planning an exercise as described herein. Any other software, programs or instructions described herein may also be stored and executed by computer system 200. It will be appreciated that computer code may be applied to any aspects of methods and techniques described herein.

The various methods or processes outlined herein may be coded as software that is executable on one or more processors that employ any one of a variety of operating systems or platforms. Additionally, such software may be written using any of numerous suitable programming languages and/or programming or scripting tools, and also may be compiled as executable machine language code or intermediate code that is executed on a virtual machine or a suitable framework.

In this respect, various inventive concepts may be embodied as at least one non-transitory computer readable storage medium (e.g., a computer memory, one or more floppy discs, compact discs, optical discs, magnetic tapes, flash memories, circuit configurations in Field Programmable Gate Arrays or other semiconductor devices, etc.) encoded with one or more programs that, when executed on one or more computers or other processors, implement the various embodiments of the present invention. The non-transitory computer-readable medium or media may be transportable, such that the program or programs stored thereon may be loaded onto any computer resource to implement various aspects of the present invention as discussed above.

The terms “logic”, “control logic”, “program”, “software”, “application”, “method” and/or “process” are used herein in a generic sense to refer to any type of computer code or set of computer-executable instructions that can be employed to program a computer or other processor to implement various aspects of embodiments as discussed above. Additionally, it should be appreciated that according to one aspect, one or more computer programs that when executed perform methods of the present disclosure need not reside on a single computer or processor, but may be distributed in a modular fashion among different computers or processors to implement various aspects of the present invention.

Computer-executable instructions may be in many forms, such as program modules, executed by one or more computers or other devices. Generally, program modules include routines, programs, objects, components, data structures, etc. that perform particular tasks or implement particular abstract data types. Typically, the functionality of the program modules may be combined or distributed as desired in various embodiments. Such computer code or computer-executable instructions may take the form of software and/or firmware executing on one or more programmable processors, field-programmable gate arrays (FPGAs), and/or digital signal processors. All or a portion of such code or instructions may also alternatively be implemented in the form of hardwired circuitry on, for example, an application-specific integrated circuit (ASIC).

Also, data structures may be stored in non-transitory computer-readable storage media in any suitable form. Data structures may have fields that are related through location in the data structure. Such relationships may likewise be achieved by assigning storage for the fields with locations in a non-transitory computer-readable medium that convey relationship between the fields. However, any suitable mechanism may be used to establish relationships among information in fields of a data structure, including through the use of pointers, tags or other mechanisms that establish relationships among data elements.

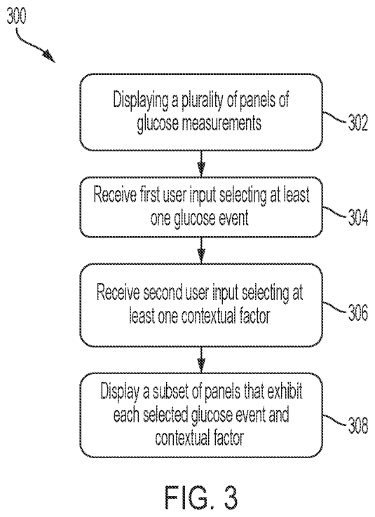

FIG. 3 is a flowchart depicting an exemplary process 300 for displaying logged data pertaining to a person with diabetes, according to some embodiments. In general, process 300 may be implemented on a suitable computing device having a user interface that includes a display screen that visually displays information, and one or more user input devices, such as a touch screen, keyboard, mouse, microphone, camera, or some other input device. In some embodiments, process 300 may be implemented by HCP system 102, health IT system 118, mobile device 104, C3 servers 140-146, administrator-portal system 148, or any combination of the aforementioned systems, servers, or devices. References to “displaying” data on a display screen may refer to displaying data on display 120 of the HCP system 102, on display 122 of mobile device 104, or on a display associated with health IT system 118, any or all of C3 servers 140-146, and/or administrator-portal system 148.

At step 302, process 300 displays on the display screen of the computing device a plurality of panels, each panel displaying one or more glucose measurements for a person with diabetes recorded at different time periods. As used herein, a “panel” may refer to a group or collection of visual data including text, symbols, and/or graphics generated by a processor for display on a defined area of a visual display screen. The defined area of a “panel” need not be square or rectangular but may take any shape; furthermore, the defined area of a “panel” is not limited to a subset of the display screen but may, in some embodiments, encompass the entire visible area of the display screen. A “panel” may comprise multiple “sub-panels”, as discussed in further detail herein. Also as used herein, “glucose measurements” may refer to measurements taken of the person's blood glucose or interstitial glucose levels. Such glucose measurements may have been logged by mobile device 104 based on manual user input from patient 124, or from data received from glucose meter 126 via communication link 128. Also as used herein, a time at which a glucose measurement is “recorded” may refer to a time at which the glucose measurement was detected or measured by a glucose sensor, a time at which the glucose measurement was transmitted to a glucose sensor and/or received by a mobile device (e.g., a smartphone), a time at which the glucose measurement was input into a mobile device by a user, and/or a time at which the glucose measurement was uploaded by the mobile device to a cloud server.

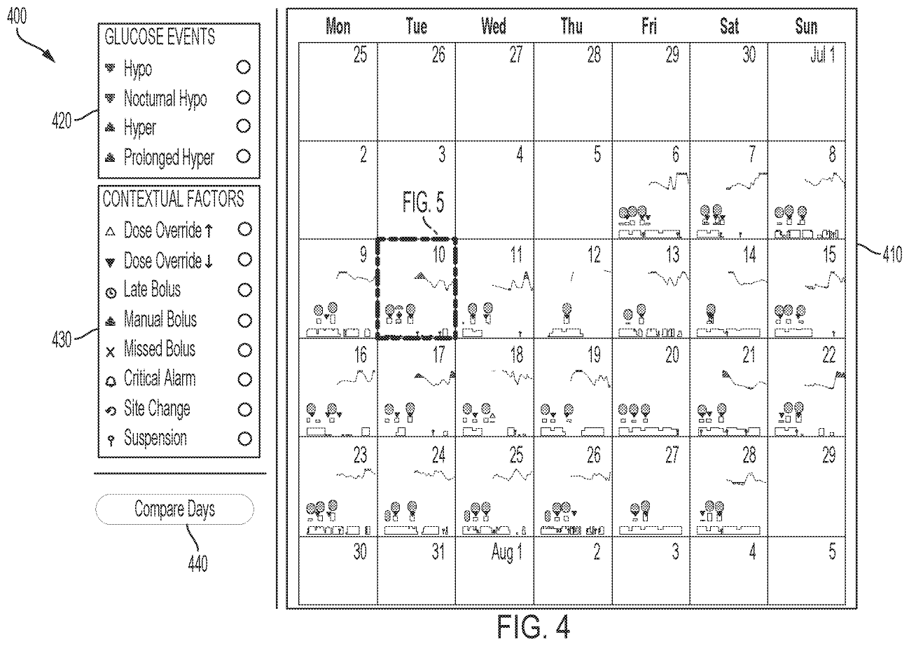

FIG. 4 provides one example of a display screen 400 displaying the aforementioned plurality of panels including one or more glucose measurements for the person with diabetes, according to some embodiments. In this example, display screen 400 includes a plurality of panels collectively labeled 410, each panel pertaining to a single day in the month of July. Each panel displays one or more glucose measurements for the person recorded during that corresponding day. While each panel in the embodiment displayed in FIG. 4 correspond to a unique day, other embodiments are possible in which each panel could correspond to a time window of any suitable length (e.g., one hour, half a day, multiple days, one week, one month, and the like). In some embodiments, the length of the time period to which each panel corresponds may be selectable by a user. Regardless of the length of the time window to which each panel corresponds, each panel preferably displays time windows that are unique and non-overlapping.

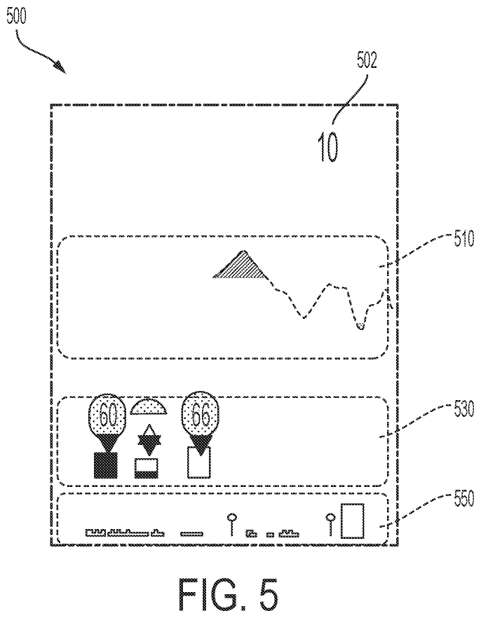

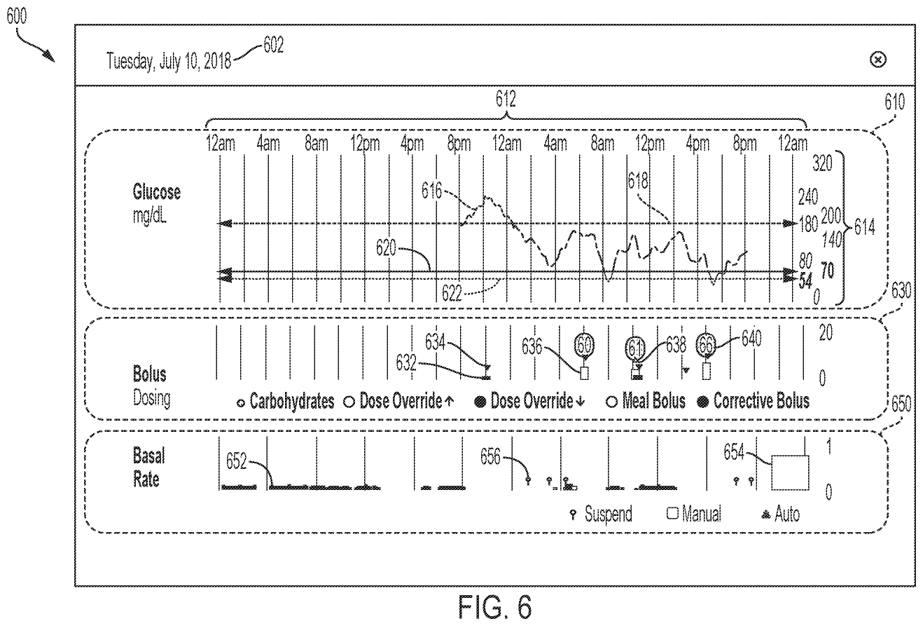

For clarity, FIG. 5 provides a close-up view of panel 500, which pertains to one of the days depicted in FIG. 4. Panel 500 includes a date label 502, which indicates that panel 500 is displaying data recorded on July 10. Panel 500 also includes a glucose measurement sub-panel 510 which includes one or more glucose measurements taken throughout the day, depicted in the form of a line graph (where the horizontal axis indicates the passage of time over the day, and the vertical axis indicates the glucose level). Panel 500 also includes a bolus dosing sub-panel 530 which depicts data regarding bolus doses recorded as being administered to the person throughout that day. Panel 500 also includes a basal dosing sub-panel 550, which depicts data regarding basal doses recorded as being administered to the person throughout that day. Glucose measurement sub-panel 510, bolus dosing sub-panel 530, and basal dosing sub-panel 550 may display data using the same or similar format discussed in further detail below, with regard to FIG. 6.

FIG. 6 provides an alternative, more detailed panel 600 pertaining to the same day, i.e., July 10. In some embodiments, panel 600 may be displayed in place of the simpler panel 500 on display screen 400. In other embodiments, panel 600 may be displayed when a user selects panel 500, e.g., by clicking on it, touching it, or otherwise indicating that the user wishes to see more detail regarding panel 500. Similar to panel 500, panel 600 also includes a data label 602, which indicates that panel 600 is displaying data recorded on Jul. 10, 2018. Panel 600 also includes a glucose measurement sub-panel 610 analogous to glucose measurement sub-panel 510, a bolus dosing sub-panel 630 analogous to bolus dosing sub-panel 530, and a basal dosing sub-panel 650 analogous to basal dosing sub-panel 550.

Glucose measurement sub-panel 610 includes a horizontal time axis 612 which indicates the passage of time from 12 am in the morning until 12 am the next day. Sub-panel 610 also includes a vertical axis 614 which indicates glucose levels, e.g., in units of mg/dL. Sub-panel 610 includes a glucose measurement line 616 which displays glucose measurements taken of the person at different times on July 10. For example, such glucose measurements may be taken by glucose meter 126 and received and logged by mobile device 104 (see FIG. 1). As previously discussed, glucose meter 126 may be a Continuous Glucose Monitor (CGM), a Blood Glucose Monitor (BGM) or a Flash Glucose Monitor (FGM). A CGM and/or a FGM may enable numerous measurements to be taken over the course of the day—conversely, a BGM may only allow glucose measurements to be taken less frequently (e.g., only 3-4 times a day). Glucose measurement sub-panel 610 further includes an upper glucose threshold line 618 which visually indicates the threshold between a normal glucose level and a hyperglycemic glucose level. In this embodiment, upper glucose threshold line 618 is set at 180 mg/dL, though other settings may also be used. When the person's glucose levels are above the glucose levels indicated by upper glucose threshold line 618, the person is considered to be having a hyperglycemic episode or event. Similarly, glucose measurement sub-panel 610 also includes a lower glucose threshold line 620. Lower glucose threshold line 620 indicates the threshold between a normal glucose level and a hypoglycemic glucose level. In this embodiment, lower glucose threshold line 620 is set at 70 mg/dL, though other settings may also be used. Glucose measurement sub-panel 610 further includes an extreme lower glucose threshold line 622. Extreme lower glucose threshold line 622 indicates the threshold between a hypoglycemic glucose level and an extremely or critically hypoglycemic glucose level. When the person's glucose levels are between the glucose levels indicated by lower glucose line 620 and extreme lower glucose threshold line 622, the person is considered to be having a hypoglycemic episode or event. When the person's glucose levels are below extreme lower glucose threshold line 622, the person is considered to be having an extreme or critical hypoglycemic episode or event. As discussed in further detail herein, hyperglycemic events, hypoglycemic events, and extreme/critical hypoglycemic events are examples of glucose events.

Bolus dosing sub-panel 630 depicts a plurality of blocks 632 indicating corrective bolus doses administered to the person, and a plurality of blocks 636 indicating meal bolus doses administered to the person. As used herein, a “meal” bolus dose may refer to a bolus dose taken to counteract a rise or expected rise in glucose levels resulting from a meal. A “corrective” bolus dose may refer to a bolus dose not taken in association with a specific meal, but in order to counteract an abnormally high glucose level. Each block 632 and block 636 is arranged horizontally to correspond with the time scale depicted on horizontal time axis 612, such that each block's horizontal position relative to horizontal time axis 612 indicates the time at which that block's bolus dose was administered to the person. Each block's height is scaled in proportion to the amount of insulin administered at that bolus. In the example depicted in FIG. 6, meal bolus blocks 636 are depicted as white blocks while corrective bolus blocks 632 are depicted as solid black blocks. However, these two types of meal blocks may also be visually distinguished from each other by using different colors, hashing, intensity levels, or other visually distinguishing features. In some embodiments, bolus dosing sub-panel 630 may not differentiate between meal bolus doses and corrective bolus doses and may simply display a single type of block for all types of bolus doses.

Bolus dosing sub-panel 630 may further include one or more downward dose override indicators 634 (shaped as a downward pointing triangle shaded in solid black) and/or one or more upward dose override indicators 638 (shaped as an upward pointing triangle shaded in white). A downward dose override indicator 634 positioned on top of a particular bolus dose block indicates that a computing device (e.g., a device having a bolus advisor or bolus calculator implemented thereon) had recommended that the person take more insulin on a particular bolus based on various factors, such as the person's glucose levels, carb intake, and/or amount of active insulin from previous boluses—however, the person took less insulin on that bolus than recommended (e.g., the device had recommended the person take 12 units of insulin, but the person only took 10 units). An upward dose override indicator 638 positioned on top of a particular bolus dose block indicates that the computing device had recommended that the person take less insulin on a particular bolus (again based on various factors), but the person took more insulin on that bolus than recommended (e.g., the device had recommended the person take 10 units of insulin, but the person took 12 units). As discussed in further detail below, a downward dose override or an upward dose override are examples of contextual factors.

Bolus dosing sub-panel 630 may further include one or more carbohydrate indicators 640. The carbohydrate indicators may indicate the number of carbohydrates that the person ingested in a meal (e.g., 60 grams, 61 grams, and 66 grams at around 6 am, 10 am, and 4 pm respectively in the example depicted in FIG. 6). As with the other elements in sub-panel 630, the horizontal placement of these carbohydrate indicators 640 corresponds with the time scale depicted on horizontal time axis 612.

Basal dosing sub-panel 650 depicts a plurality of blocks 652 indicating auto basal micro-boluses administered to the person, and a plurality of blocks 654 indicating manual basal micro-boluses administered to the person. As used herein, an “auto” basal micro-bolus may refer to a micro-bolus (e.g., a small bolus of basal insulin, in the range of 0.025 units to 1 units) determined and administered automatically to the person by an automated insulin delivery device, such as an insulin pump. In some embodiments, such an “auto” basal micro-bolus may be determined and administered without any manual input by the person at the time the bolus is administered, e.g., without requiring that the person provide any instruction, respond to any prompt, or provide any information or confirmation. Instead, such “auto” basal micro-bolus may be automatically determined by the insulin delivery device based on pre-programmed parameters provided by the person with diabetes, a caregiver, or a health care provider. As used herein, a “manual” micro-bolus may refer to a micro-bolus manually requested by the person. Each block 652 and 654 is arranged horizontally to correspond with the time scale depicted on horizontal time axis 612, such that each block's position along horizontal time axis 612 indicates the time at which that block's micro-bolus was administered to the person. Each block's height is scaled in proportion to the amount of insulin administered during that micro-bolus. In the example depicted in FIG. 6, manual basal micro-boluses 654 are depicted as white blocks while auto basal micro-boluses 652 are depicted as solid black blocks. However, these two types of basal micro-bolus blocks may also be visually distinguished from each other by using different colors, hashing, intensity levels, or other visually distinguishing features. In some embodiments, basal dosing sub-panel 650 may not differentiate between auto- and manual basal micro-boluses at all and may simply display a single type of block for all types of basal micro-boluses.

Basal dosing sub-panel 650 may further include a symbol or indicator 656 that indicates the occurrence of a Predictive Low Glucose Suspend (PLGS). A PLGS occurs when an automated insulin delivery device (e.g., an insulin pump) detects that the person's glucose levels are below or trending towards a hypoglycemic condition, in which case the delivery device may automatically suspend delivery of basal insulin until the person's glucose levels stabilize. Each PLGS indicator 656 is arranged horizontally to correspond with the time scale 612, such that each indicator's position along horizontal time axis 612 indicates the time at which that PLGS event occurred. As discussed in further detail below, a PLGS is an example of a contextual factor.

Although these features are not explicitly labeled in FIG. 5 for ease of readability, glucose measurement sub-panel 510 may be configured similarly to glucose measurement sub-panel 610 and may incorporate the same or similar horizontal and vertical axes, threshold lines and glucose measurement indicators. Bolus dosing sub-panel 530 may be configured similarly to bolus dosing sub-panel 630 and may incorporate the same or similar dosing blocks, symbols, and carbohydrate indicators. Basal dosing sub-panel 550 may be configured similarly to basal dosing sub-panel 650 and may incorporate the same or similar dosing blocks and indicators.

Returning to FIG. 3, at step 304, process 100 receives first user input selecting at least one of a plurality of glucose events. Display screen 400 on FIG. 4 shows one exemplary panel 420 through which the user may provide input selecting at least one of a plurality of glucose events. Glucose event panel 420 displays a list of exemplary glucose events, such as “Hypo” (or hypoglycemic episode), “Nocturnal Hypo” (or nocturnal hypoglycemic episode), “Hyper” (or hyperglycemic episode), or “Prolonged Hyper” (or prolonged hyperglycemic episode). A hypoglycemic episode may be defined as any stretch of time during which the person's glucose levels dropped below 70 mg/dL. A nocturnal hypoglycemic episode may be defined as any hypoglycemic episode that occurs between the hours of midnight and 6 am. A hyperglycemic episode may be defined as any stretch of time during which the person's glucos e levels increased above 180 mg/dL. A prolonged hyperglycemic episode may be defined as any hyperglycemic episode that persisted for more than 4 hours. It should be understood that the previously described glucose thresholds and time periods are exemplary only and may be configured differently according to different embodiments. For example, the threshold for a hypoglycemic episode may alternatively be set at any glucose level between 60-80 mg/dL, and the threshold for a hyperglycemic episode may alternatively be set at any glucose level between 170-190 mg/dL. Similarly, the time bounds for a nocturnal hypoglycemic episode may be set for any time period occurring between, for example, the hours of 11 pm and 7 am, and the threshold time period for a prolonged hyperglycemic episode may be set at any duration between 3 and 5 hours.

Each of the aforementioned glucose events is associated with a user-selectable radio button. A user, such as a health care professional (HCP), a care provider, the person with diabetes, and/or the person's caregiver or loved one, may select one or more of these glucose events by selecting these radio buttons.

Returning to FIG. 3, at step 306, process 300 receives second user input selecting at least one of a plurality of contextual factors. Display screen 400 on FIG. 4 shows one exemplary panel 430 through which the user may provide input selecting at least one of a plurality of contextual factors. Contextual factors panel 430 displays a list of exemplary contextual factors, such as upward dose override, downward dose override, late bolus, manual bolus, missed bolus, critical alarm, site change, and suspension. As previously described in relation to upward dose override indicator 638 (see FIG. 6), an upward dose override event may include an event in which the computing device had recommended that the person take less insulin on a particular bolus (based on various factors, such as the person's glucose levels, carb intake, and/or amount of active insulin from previous boluses), but the person took less insulin on that bolus than recommended. For example, the device may have recommended that the person take 12 units of insulin, but the person only took 10 units on that bolus. Also as previously described in relation to downward dose override indicator 634 (see FIG. 6), a downward dose override event may be an event in which the computing device had recommended that the person take more insulin on a particular bolus (again, based on various factors), but the person took less insulin on that bolus than recommended.

A missed bolus may include a situation where a person with diabetes that requires meal-time insulin has ingested food (such ingestion of food also referred to herein as a “meal event”) without taking an insulin bolus to compensate for an increase in glucose levels resulting from, or expected to result from, the ingested food. A “meal event” or “food” can include any type of food, drink, or meal that can be expected to result in an increase in the user's glucose levels, including without limitation breakfast, lunch, dinner, any snacks, and/or any drinks. A late bolus may include a situation where a person with diabetes that requires meal-time insulin ingests food at a meal event, but takes an insulin bolus too late in time to appropriately compensate for the meal event. This may result in an undesirable spike in the person's glucose levels before the insulin bolus takes effect. Missed boluses and late boluses may be detected by monitoring the person's glucose levels and/or glucose level trends over time and analyzing said glucose levels in conjunction with a log of the person's insulin boluses (e.g., the time and amount of previously administered insulin boluses).

Several exemplary and illustrative methods for detecting missed boluses and/or late boluses based on glucose measurements and insulin dosing information will now be discussed. At least one processor of the aforementioned computer systems may execute software and/or firmware code to implement these methods.

1. Glucose Increase Threshold Method

One exemplary method for detecting missed boluses, referred to herein as the “Glucose Increase Threshold” method, is to determine that the person may have missed an insulin bolus if the following conditions are fulfilled:

(i) the person's glucose level increases by more than a maximum allowable glucose increase threshold (ΔGₘₐₓ, e.g., 20-60 mg/dL) within a predetermined glucose-consideration time window (T_{G}, e.g., 5-10 minutes) of the current time; and
(ii) the person has not taken an insulin bolus within a predetermined bolus-consideration time period (T_{B}, e.g., 2 hours) of the current time.

The parameters ΔGₘₐₓ, T_{G}, and T_{B} may be adjusted to make the Glucose Increase Threshold method more or less sensitive. For example, increasing the maximum allowable glucose increase threshold (ΔGₘₐₓ) would decrease the sensitivity, while decreasing ΔGₘₐₓ would increase the sensitivity. Increasing the glucose-consideration time window (T_{G}) would increase the sensitivity, while decreasing T_{G} would decrease the sensitivity. Increasing the bolus-consideration time window (T_{B}) would decrease the sensitivity, while decreasing T_{B} would increase the sensitivity.

The Glucose Increase Threshold method may also be used to determine that the person may have taken a bolus late (i.e., that a late bolus event has occurred). The same conditions (i) and (ii) described above may be used to determine whether the person took a bolus late. In some embodiments, a shorter bolus-consideration time period T_{B} may be used to detect late boluses than to detect missed boluses.

2. Glucose Rate-of-Change (“ROC”) Threshold Method

Another exemplary method for detecting missed boluses, referred to herein as the “Glucose ROC Threshold” method, is to determine that the person may have missed an insulin bolus if the following conditions are satisfied:

(i) the person's glucose levels exhibit a rate-of-change (ROC_{G}) that is greater than a maximum allowable glucose rate-of-change threshold (ROCₘₐₓ, e.g., 2 mg/dL/hr); and
(ii) the person has not taken an insulin bolus within a predetermined bolus-consideration time period (T_{B}, e.g., 2 hours) of the current time.

ROC_{G} may be provided or calculated by some commercially available Continuous Glucose Monitors (CGM), such as the G6 CGM sensor manufactured and sold by Dexcom, Inc. For example, if a glucose sensor records three consecutive glucose readings, each spaced no more than 5 minutes apart from its closest neighbor reading in time, ROC_{G} may be calculated by dividing the difference between the last and the first glucose reading by the amount of time that has elapsed between the first and the last glucose reading. Other methods or devices for calculating ROC_{G} may also be used.

The parameters ROCₘₐₓ and T_{B} may be adjusted to make the Glucose ROC Threshold method more or less sensitive. For example, increasing ROCₘₐₓ would decrease the sensitivity, while decreasing ROCₘₐₓ would increase the sensitivity. Increasing the bolus-consideration time window (T_{B}) would decrease the sensitivity, while decreasing T_{B} would increase the sensitivity.

The Glucose ROC Threshold method may also be used to determine that the person may have taken a bolus late (i.e., that a late bolus event has occurred). The same conditions (i) and (ii) described above may be used to determine whether the person took a bolus late. In some embodiments, a shorter bolus-consideration time period T_{B} may be used to detect late boluses than to detect missed boluses.

3. Absolute Glucose Level Threshold Method

Yet another exemplary method for detecting missed boluses, referred to herein as the “Absolute Glucose Level Threshold” method, is to determine that the person may have missed an insulin bolus if the following conditions are satisfied:

(i) the person's glucose levels exceed an absolute glucose level threshold (Gₘₐₓ, e.g., 180 mg/dL); and
(ii) the person has not taken an insulin bolus within a predetermined bolus-consideration time period (T_{B}, e.g., 2 hours)

The parameters Gₘₐₓ and T_{B} may be adjusted to make the Absolute Glucose Level Threshold method more or less sensitive. For example, increasing the maximum allowable glucose threshold (Gₘₐₓ) would decrease the sensitivity, while decreasing Gₘₐₓ would increase the sensitivity. Increasing the bolus-consideration time window (T_{B}) would decrease the sensitivity, while decreasing T_{B} would increase the sensitivity.

The Absolute Glucose Level Threshold method may also be used to determine that the person may have taken a bolus late (i.e., that a late bolus event has occurred). The same conditions (i) and (ii) described above may be used to determine whether the person took a bolus late. In some embodiments, a shorter bolus-consideration time period T_{B} may be used to detect late boluses than to detect missed boluses.

A manual bolus may include a situation where a person with diabetes provides manual user input instructing an insulin delivery device (e.g., an insulin pump) to provide a specified bolus of insulin at a specified time. As previously discussed, a manual bolus may be contrasted with an “auto” basal micro-bolus determined and administered automatically to the person by an automated insulin delivery device.

A critical alarm may include a situation where an insulin delivery device detects a technical error or problem with its operation. Such errors or problems include an occlusion in the fluid pathway between an insulin reservoir and a site of insulin administration, damage or incorrect operation of some component of the insulin delivery device (e.g., the pump, dose determination sensors, etc.), low battery power, lack of connectivity with a mobile device and/or glucose sensor (e.g., due to electromagnetic interference, or too much distance between the devices), excessive heat or cold, insufficient insulin in the delivery device's reservoir, or other types of technical issues that prevent smooth, error-free, and/or efficient operation of the insulin delivery device.

A site change may include a situation where a user temporarily removes the insulin delivery device in order to change the injection site, e.g., from the person's abdomen to the person's back. Site changes may be detected based on manual user input indicating that the user is changing his/her injection site. In some embodiments, a site change may be inferred each time the insulin delivery device is powered off and on.

A suspension may include the occurrence of a Predictive Low Glucose Suspend (PLGS), as previously described in relation to FIG. 6.

Returning to FIG. 3, at step 308, process 300 displays on a display screen a subset of panels from the plurality of panels displayed at step 302. Each panel in the subset of panels satisfies at least two conditions: (i) each panel displays one or more glucose measurements that exhibit each of the glucose events selected at step 304, and (ii) each panel displays one or more glucose measurements recorded during a time period in which the patient exhibited each of the at least one contextual factors selected at step 306. Each panel in the subset of panels may be visually highlighted or emphasized in some way, while each panel not in the subset of panels may be visually de-emphasized or not displayed at all. Methods of visually highlighting panels include increasing the panels' size, changing the panels' color, changing or enlarging the panels' font, adding visual elements such as outlining and symbols, or otherwise changing the appearance of the highlighted panels. Conversely, methods of visually de-emphasizing panels include fading, obscuring, shrinking, removing entirely, or de-saturating said de-emphasized panels.

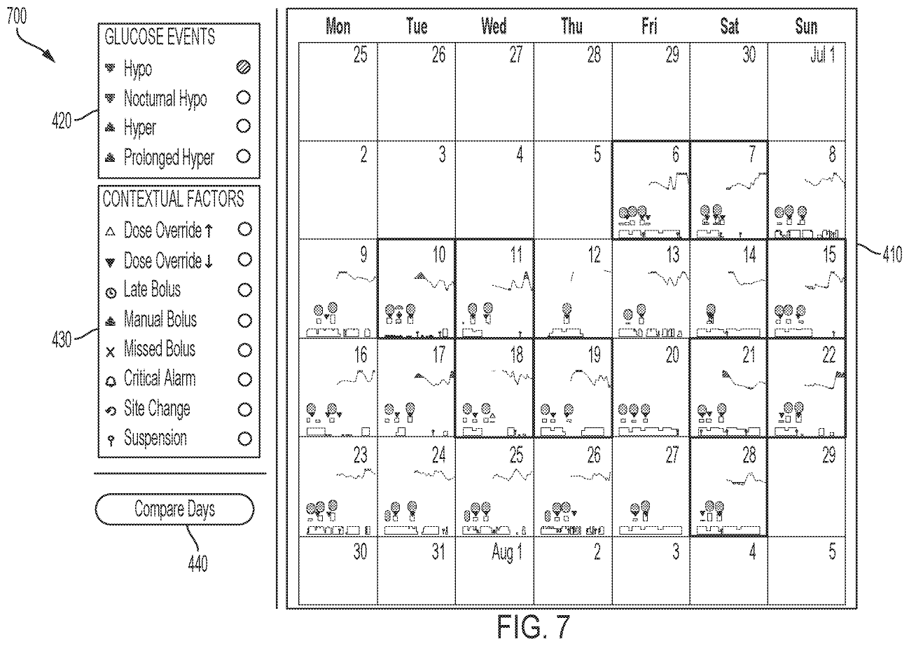

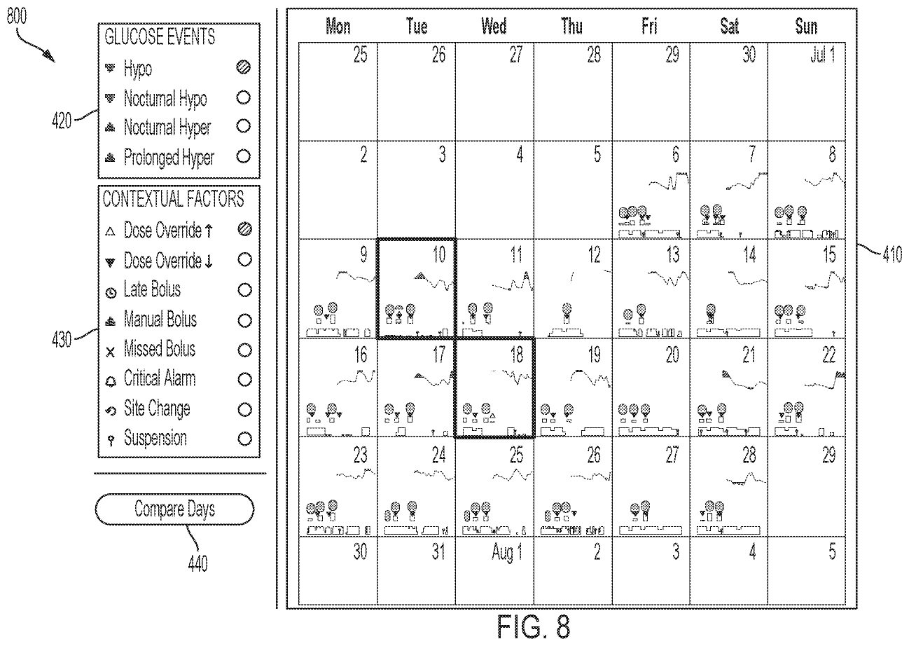

FIG. 7 and FIG. 8 provide an example of step 308 in operation, according to some embodiments. FIG. 7 shows a display screen 700 in which the user has selected the glucose event “hypo” (e.g., hypoglycemic event) within glucose event panel 420. In response, display screen 700 visually emphasizes the subset of panels that displays one or more glucose measurements that exhibit a hypoglycemic event. In this example, that subset of panels includes the panels pertaining to the following days: July 6, 7, 10, 11, 15, 18, 19, 21, 22, and 28. In this case, the visual emphasis may take the form of a thick black border around all emphasized panels. All other panels in the plurality of panels 410 are grayed out in the figure, illustrating that these panels are visually de-emphasized.

FIG. 8 shows a display screen 800 in which a user has selected the glucose event “hypo” (e.g., hypoglycemic event) within glucose event 420, and has also selected the contextual factor “upward dose override” in contextual factors panel 430. In response, display screen 800 visually emphasizes the subset of panels that both displays one or more glucose measurements that exhibit a hypoglycemic event, and which pertain to a time period in which the patient exhibited the contextual factor “upward dose override.” In this example, that subset of panels includes the panels pertaining to the following days: July 10 and 18 only. Again, the visual emphasis in this example takes the form of a thick black border around all emphasized panels. All other panels in the plurality of panels 410 are grayed out in the figure, illustrating that these panels are visually de-emphasized.

In this way, the disclosed systems, methods and processes allow the user to filter logged data pertaining to a person with diabetes according to both (i) glucose events and (ii) contextual factors. Panels displaying data that exhibit both the selected glucose events and the contextual factors are visually highlighted and/or emphasized. This allows the user, such as a HCP, to quickly identify, analyze, and/or compare time periods in which the person with diabetes exhibited not just one type of event, but different combinations of events. For example, the user may quickly determine by filtering for both hypoglycemic events and upward dose overrides whether the person with diabetes has a tendency to manually direct his/her automatic insulin delivery device to deliver too much insulin, thus sending the person into hypoglycemia. If filtering for both hypoglycemic events and upward dose overrides reveal that the person has a consistent tendency to manually and inappropriately increase his/her dose, this may be the basis of a discussion between the person and his/her health care provider to explore root causes and solutions for this tendency.

When none of the panels of the plurality of panels 410 are visually emphasized, the “Compare Days” button 440 may be grayed out or deactivated, as shown in FIG. 4. However, whenever two or more of the panels in the plurality of panels 410 are selected or visually emphasized, the “Compare Days” button 440 may become active, as shown in FIGS. 7 and 8. When a user selects the activated “Compare Days” button 440, the system may display a comparison of the selected or visually emphasized panels.

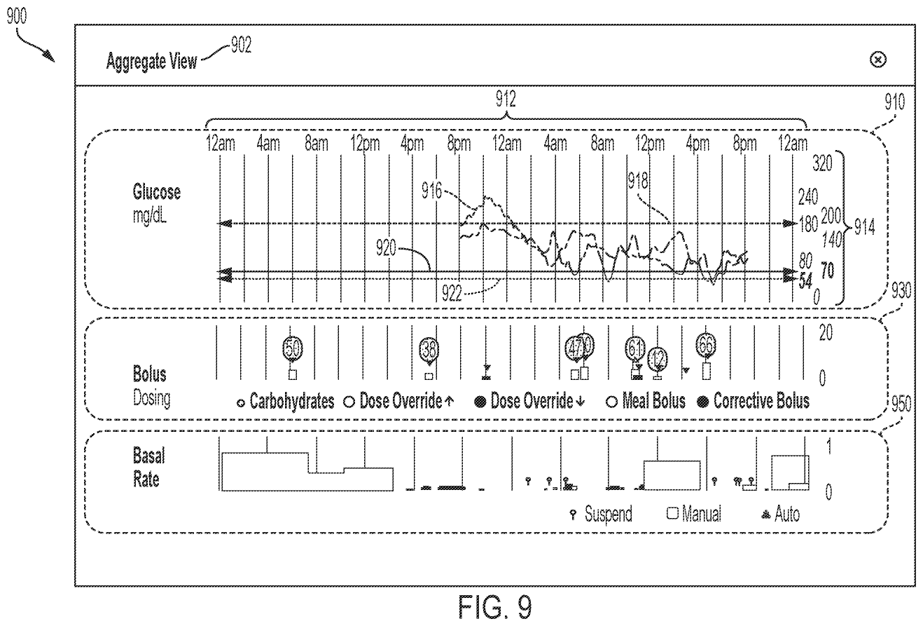

FIG. 9 shows an exemplary panel 900 for comparing data from multiple panels, according to some embodiments. Panel 900 includes a data label 902 (in approximately the same position as data label 602 in FIG. 6), which indicates that panel 900 is displaying an “Aggregate View”, e.g., a view in which data from multiple panels have been “aggregated” for comparison or analysis. Panel 900 also includes a glucose measurement sub-panel 910 analogous to glucose measurement sub-panel 610, a bolus dosing sub-panel 930 analogous to bolus dosing sub-panel 630, and a basal dosing sub-panel 950 analogous to basal dosing sub-panel 650.

Glucose measurement sub-panel 910 includes a horizontal time axis 912 that is analogous to horizontal time axis 612, which indicates the passage of time from 12 am in the morning until 12 am the next day. Sub-panel 910 also includes a vertical axis 914 that is analogous to vertical axis 614, which indicates glucose levels in units of mg/dL. Sub-panel 910 also includes two or more glucose measurement lines 916. Each glucose measurement line displays multiple glucose measurements taken of the person at different times on the selected time periods. In this example, panel 900 is comparing data from the two panels that were visually emphasized in FIG. 8, i.e., the panels pertaining to July 10 and July 18. As a result, glucose measurement sub-panel 910 includes two glucose measurement lines 916, one pertaining to July 10 and the other pertaining to July 18. Glucose measurement sub-panel 910 also includes an upper glucose threshold line 918 analogous to upper glucose threshold line 618, a lower glucose threshold line 920 analogous to lower glucose threshold line 620, and an extreme lower glucose threshold line 922 analogous to extreme lower glucose threshold line 622.

Bolus dosing sub-panel 930 is analogous to bolus dosing sub-panel 630. However, whereas sub-panel 630 only displays data pertaining to one time period, sub-panel 930 may display data pertaining to each time period that corresponds to one of the selected or visually emphasized panels (in this example, the panels pertaining to July 10 and July 18). Similar to bolus dosing sub-panel 630, the displayed data may comprise meal boluses blocks 632, corrective bolus blocks 636, downward dose override indicators 634, upward dose override indicators 638, and/or carbohydrate indicators 640.

Basal dosing sub-panel 950 is analogous to bolus dosing sub-panel 640. Similarly, however, whereas sub-panel 650 only displays data pertaining to one time period, sub-panel 950 may display data pertaining to each time period that corresponds to one of the selected or visually emphasized panels (e.g., July 10 and July 18). This displayed data may comprise auto basal micro-bolus blocks 652, auto basal micro-bolus blocks 654, and/or one or more PLGS symbols or indicators 656.

The terms “first”, “second”, “third” and the like, whether used in the description or in the claims, are provided for distinguishing between similar elements and not necessarily for describing a sequential or chronological order. It is to be understood that the terms so used are interchangeable under appropriate circumstances (unless clearly disclosed otherwise) and that the embodiments of the disclosure described herein are capable of operation in other sequences and/or arrangements than are described or illustrated herein. In particular, while FIG. 3 describes the receiving of “first” user input at step 304 and the receiving of “second” user input at step 306, it is to be understood that, both in the specification and in the claims, the “second” user input described at step 306 may be received before, at the same time, or after the “first” user input described at step 304 is received.

While this invention has been described as having exemplary designs, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

1. A method for displaying selected patient data on a display screen of a computing device, the method comprising:
displaying, on the display screen of the computing device, a plurality of panels, each panel associated with a unique time window and displaying one or more glucose measurements for a patient recorded during the unique time window;
receiving a first user input selecting at least one glucose event type of a plurality of glucose event types;
receiving a second user input selecting at least one contextual factor type of a plurality of contextual factor types; and
displaying, on the display screen in response to receiving the first and second user inputs, a subset of panels from the plurality of panels that are visually distinguished over other panels from the plurality of panels not included in the subset, wherein each panel in the subset of panels displays at least one glucose measurement that exhibits the selected at least one glucose event type and was recorded during a time period in which the patient experienced the selected at least one contextual factor type.

2. The method of claim 1, further comprising displaying, on the display screen of the computing device, the plurality of glucose event types and the plurality of contextual factor types separate from the plurality of panels.

3. The method of claim 1, wherein displaying the subset of panels that are visually distinguished over other panels from the plurality of panels not included in the subset comprises visually de-emphasizing panels from the plurality of panels that do not belong to the subset by fading, obscuring, shrinking, or de-saturating the de-emphasized panels on the display screen.

4. The method of claim 1, wherein displaying the subset of panels that are visually distinguished over other panels from the plurality of panels not included in the subset comprises removing, from the display screen in response to receiving the first and second user inputs, panels from the plurality of panels that do not belong to the subset of panels.

5. The method of claim 1, wherein displaying the subset of panels that are visually distinguished over other panels from the plurality of panels not included in the subset comprises visually emphasizing the subset of panels on the display screen by changing a color of said subset of panels, adding a border around said subset of panels, adding a symbol to each panel of said subset of panels, or increasing a size of each panel of said subset of panels.

6. The method of claim 1, wherein the plurality of glucose events comprise at least one of a hypoglygemic event, a nocturnal hypoglycemic event, a hyperglycemic event, and a prolonged hyperglycemic event.

7. The method of claim 1, wherein the plurality of contextual factors comprise at least one of a user override of an automatic dose increase, a user override of an automatic dose decrease, a late bolus, a manual bolus, a missed bolus, a critical pump alarm, a change of an infusion site, and a suspension of an automatic infusion dosing algorithm.

8. The method of claim 1, wherein each panel of the plurality of panels displays one or more glucose measurements for a patient recorded during a different day.

9. The method of claim 1, wherein each panel displays one or more glucose measurements recorded by at least one of a blood glucose monitor (BGM), a continuous glucose monitor (CGM), and a flash glucose monitor (FGM).

10. A computing device comprising:
a display screen;
at least one processor; and
non-transitory computer-readable media storing computer-executable instructions that, when executed by the at least one processor, are operable to cause the at least one processor to:
display on the display screen a plurality of panels, each panel associated with a unique time window and displaying one or more glucose measurements for a patient recorded during the unique time window;
receive a first user input selecting at least one glucose event type of a plurality of glucose event types;
receive a second user input selecting at least one contextual factor type of a plurality of contextual factor types; and
display, on the display screen in response to receiving the first and second user inputs, a subset of panels from the plurality of panels that are visually distinguished over other panels from the plurality of panels not included in the subset, wherein each panel in the subset of panels displays at least one glucose measurement that exhibits the selected at least one glucose event type and was recorded during a time period in which the patient experienced the selected at least one contextual factor type.

11. The computing device of claim 10, wherein the at least one processor is further configured to display, on the display screen of the computing device, the plurality of glucose event types and the plurality of contextual factor types separate from the plurality of panels.

12. The computing device of claim 10, wherein displaying the subset of panels that are visually distinguished over other panels from the plurality of panels not included in the subset comprises visually de-emphasizing panels from the plurality of panels that do not belong to the subset by fading, obscuring, shrinking, or de-saturating the de-emphasized panels on the display screen.

13. The computing device of claim 10, wherein displaying the subset of panels that are visually distinguished over other panels from the plurality of panels not included in the subset comprises removing, from the display screen in response to receiving the first and second user inputs, panels from the plurality of panels that do not belong to the subset of panels.

14. The computing device of claim 10, wherein displaying the subset of panels that are visually distinguished over other panels from the plurality of panels not included in the subset comprises visually emphasizing the subset of panels on the display screen by changing a color of said subset of panels, adding a border around said subset of panels, adding a symbol to each panel of said subset of panels, or increasing a size of each panel of said subset of panels.

15. The computing device of claim 10, wherein the plurality of glucose events comprise at least one of a hypoglygemic event, a nocturnal hypoglycemic event, a hyperglycemic event, and a prolonged hyperglycemic event.

16. The computing device of claim 10, wherein the plurality of contextual factors comprise at least one of a user override of an automatic dose increase, a user override of an automatic dose decrease, a late bolus, a manual bolus, a missed bolus, a critical pump alarm, a change of an infusion site, and a suspension of an automatic infusion dosing algorithm.

17. The computing device of claim 10, wherein each panel of the plurality of panels displays one or more glucose measurements for a patient recorded during a different day.

18. The computing device of claim 10, wherein each panel displays one or more glucose measurements recorded by at least one of a blood glucose monitor (BGM), a continuous glucose monitor (CGM), and a flash glucose monitor (FGM).

19. Non-transitory computer-readable media storing computer-executable instructions that, when executed by one or more processors, are operable to cause the one or more processors to:
display, on a display screen of a computing device, a plurality of panels, each panel associated with a unique time window and displaying one or more glucose measurements for a patient recorded during the unique time window;
receive a first user input selecting at least one glucose event type of a plurality of glucose event types;
receive a second user input selecting at least one contextual factor type of a plurality of contextual factor types; and
display, on the display screen in response to receiving the first and second user inputs, a subset of panels from the plurality of panels that are visually distinguished over other panels from the plurality of panels not included in the subset, wherein each panel in the subset of panels displays at least one glucose measurement that exhibits the selected at least one glucose event type and was recorded during a time period in which the patient experienced the selected at least one contextual factor type.

20. The non-transitory computer-readable media of claim 19, wherein the computer-executable instructions, when executed by the one or more processors, are further operable to cause the one or more processors to display, on the display screen of the computing device, the plurality of glucose event types and the plurality of contextual factor types separate from the plurality of panels.

21. The non-transitory computer-readable media of claim 19, wherein displaying the subset of panels that are visually distinguished over other panels from the plurality of panels not included in the subset comprises visually de-emphasizing panels from the plurality of panels that do not belong to the subset by fading, obscuring, shrinking, or de-saturating the de-emphasized panels on the display screen.

22. The non-transitory computer-readable media of claim 19, wherein displaying the subset of panels that are visually distinguished over other panels from the plurality of panels not included in the subset comprises removing, from the display screen in response to receiving the first and second user inputs, panels from the plurality of panels that do not belong to the subset of panels.

23. The non-transitory computer-readable media of claim 19, wherein displaying the subset of panels that are visually distinguished over other panels from the plurality of panels not included in the subset comprises visually emphasizing the subset of panels on the display screen by changing a color of said subset of panels, adding a border around said subset of panels, adding a symbol to each panel of said subset of panels, or increasing a size of each panel of said subset of panels.

24. The non-transitory computer-readable media of claim 19, wherein the plurality of glucose events comprise at least one of a hypoglygemic event, a nocturnal hypoglycemic event, a hyperglycemic event, and a prolonged hyperglycemic event.

25. The non-transitory computer-readable media of claim 19, wherein the plurality of contextual factors comprise at least one of a user override of an automatic dose increase, a user override of an automatic dose decrease, a late bolus, a manual bolus, a missed bolus, a critical pump alarm, a change of an infusion site, and a suspension of an automatic infusion dosing algorithm.

26. The non-transitory computer-readable media of claim 19, wherein each panel of the plurality of panels displays one or more glucose measurements for a patient recorded during a different day.

27. The non-transitory computer-readable media of claim 19, wherein each panel displays one or more glucose measurements recorded by at least one of a blood glucose monitor (BGM), a continuous glucose monitor (CGM), and a flash glucose monitor (FGM).
